(19) United States
(12) Patent Application Publication
(10) Pub. No.: US 2026/0272564 A1
(43) Pub. Date: Sep. 17, 2026

(54) Multiple Vertebra Tracking

(71) Applicant: Fiagon GmbH, Hennigsdorf (DE)

(72) Inventor: Robert Hedermann, Berlin (DE)

(73) Assignee: Fiagon GmbH, Hennigsdorf (DE)

(21) Appl. No.: 19/338,108

(22) Filed: Sep. 24, 2025

Related U.S. Application Data
(60) Provisional application No. 63/698,756, filed on Sep. 25, 2024.

(51) Int. Cl. A61B 34/20; A61B 34/00; A61B 34/10; A61B 90/00

(52) U.S. Cl. CPC A61B 34/20; A61B 34/10; A61B 34/25; A61B 90/37; A61B 2034/105; A61B 2034/2051; A61B 2034/2055; A61B 2090/376

(57) ABSTRACT

In some embodiments, the invention refers to a method of image guided navigation. One non-limiting method comprises the steps of: equipping multiple vertebra bodies with patient localizers, segmenting the image data into vertebra body areas wherein segmentation may be performed manually or automatically, for instance by means of a segmenting neural network, and, instead of or in addition to, generating and storing a single, global coordinate system transformation during the patient registration process. In some embodiment, an individual coordinate system transformation for each localizer or vertebra body, respectively, is generated and stored.

### CROSS-REFERENCE TO RELATED APPLICATIONS

This Applications claims priority to US provisional patent application Ser. No. 63/698,756 filed Sep. 25, 2024, the entire contents of which is hereby incorporated by reference.

### BACKGROUND

The invention relates to the field of medical instruments and navigation systems and, in particular, to methods and systems for an image guided instrument navigation systems.

For an image guided instrument navigation, a trackable instrument is tracked by means of an instrument tracking system and the position and orientation of the tracked instrument is viewed on a display together with a representation of an anatomical structure of an object or a person. The representation of the anatomical structure is generated from tomographic image data of the object or person that is examined or treated by means of the tracked instrument.

The visualization of the tracked instrument together with the representation of the anatomical structure of an object or a person on the display may help a user to steer and manipulate the instrument as needed.

### SUMMARY

In a first non-limiting aspect, the invention provides a method of image guided navigation, comprising the steps of (a) generating a 3D model of a mammal form preoperatively obtained tomographic image data, (b) segmenting the 3D model for obtaining multiple 3D model segments wherein at least some 3D model segments represent rigid body parts, in particular integral bone parts such as individual vertebra bodies or bone fragments, that may move with respect to each other, (c) generating individual coordinate system transformations using a navigation system for the image guided navigation by way of registration for at least some of the 3D model segments, (d) intraoperatively tracking the position and orientation of a plurality of individual body parts of the mammal by means of the navigation system, (e) adapting a visualization of the 3D model in accordance with the respectively tracked positions and orientations of the individual body parts so as to visualize the individual body parts with their actual position and orientation relative to each other in the visualization of the 3D model, wherein the adaptation of the visualization of the 3D model comprises applying the individual coordinate system transformations to the respective tracked positions and orientations of the individual body parts, and (f) displaying the adapted visualization of the 3D model.

In various embodiments, the navigation system is an electromagnetic navigation system.

In some embodiments, the generation of individual coordinate system transformations using a navigation system for the image guided navigation by way of registration comprises intraoperative recording of image data by way of intraoperative 3D imaging or fluoroscopy imaging.

In some embodiments, the methods of the invention further comprise segmentation of the intraoperatively recorded images as represented by the intraoperatively recorded image data and performing registration between 3D model segments of the 3D model and corresponding segments of the intraoperatively recorded images.

In various embodiments, the methods of the invention further comprise attaching patient localizers to a plurality of rigid body parts, in particular to a plurality of vertebra bodies.

In some embodiments, the patient localizers are Kirschner-wires or Jamshidi-needles that each comprise a localizer coil.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The invention and further aspects shall now be described by way of exemplary embodiments with reference to the figures.

In the figures:

FIG. 1A and B illustrate how the shape of the spine depends on the posture of the patient;

FIG. 1C illustrates the decreasing accuracy of registration if a single vertebra body is tracked;

FIG. 2 further illustrates the mismatch resulting from different shapes of the spine caused by different postures of a patient;

FIG. 3A and B illustrate the segmentation of intraoperatively obtained images of a spine;

FIG. 4A shows an intraoperatively obtained gyroscopic image before surgery;

FIG. 4B shows an intraoperatively obtained gyroscopic image after surgery;

FIG. 5A and B illustrate how an individual registration of rigid body parts, in particular an individual registration of vertebra bodies may result in adapted visualization reflecting the actual position and orientation of the individual rigid body parts;

FIG. 6 illustrates how individual vertebra bodies- or individual rigid body parts in general-may be equipped individual patient localizers;

FIG. 7A and B illustrate how the individual registration of individual segments can reflect intraoperative movement of predefined areas of image data.

### DETAILED DESCRIPTION

The published patents, patent applications, websites, company names, and scientific literature referred to herein establish the knowledge that is available to those with skill in the art and are hereby incorporated by reference in their entirety to the same extent as if each was specifically and individually indicated to be incorporated by reference. Any conflict between any reference cited herein and the specific teachings of this specification shall be resolved in favor of the latter.

The instrument tracking system can be an electromagnetic (EM) tracking system that comprises at least one electromagnetic field generator that generates an electromagnetic field (EM field) in a tracking space. The electromagnetic field varies over time and position.

The trackable instrument may comprise electromagnetic field sensors (EM sensors) comprising at least one, or, alternatively, at least two sensor coils that can sense the electromagnetic field. Because the electromagnetic field is controlled to vary over time and position, electric signals captured by the sensor coils of an EM sensor can be evaluated to thus determine the position of the EM sensor- and thus the instrument-in the tracking space. The evaluation comprises measuring the voltage induced in each sensor coil. Thus, an EM sensor can provide an output signal (hereinafter also called sensor signal) that is a voltage signal that can be evaluated to find the position of the sensor inside the EM field, i.e. in the tracking space. An EM sensor with a single sensor coil can provide a sensor signal that allows determining of the position and the orientation of the EM sensor within the tracking space with respect to five degrees of freedom (5 DoF), i.e. the position in three dimensions and the orientation in two dimensions. For determining the position and the orientation of an EM sensor in all six degrees of freedom (the position in three dimensions and the orientation in three dimensions), two sensor coils are needed that are arranged at an angle with respect to one another. Typically, a 6 DoF EM sensor comprises two sensor coils each having a longitudinal axis wherein the two longitudinal axis are arranged perpendicular with respect to each other. A 6 DoF EM sensor can produce two sensor signals, i.e. one sensor signal per sensor coil.

The EM sensors comprising the sensor coils can be very small and thus can be attached to a variety of instruments, for instance surgical instruments. Thus surgical instruments can be tracked even inside a body without a line-of-sight between the sensors and the field generator.

The EM sensors comprising the sensor coils are connected to an evaluation unit that evaluates the electric signals captured by the sensor coils, for instance the voltage induced into the sensor coils by the electromagnetic field generated and emitted by the EM field generator. The EM field generator is also connected to and controlled by the evaluation unit.

In order to maintain a proper registration between the EM field and a patient for whom a tomographic 3D model may exist that can be used like a three-dimensional map of the patient, a patient localizer can be attached to a patient. The patient localizer comprises sensor coils and thus allows tracking of the position and orientation of the patient in the EM field. This enables determining a relative position of a tracked instrument relative to the (tracked) patient and in turn allows real-time visualization of the instrument in a 3D model of the patient.

EM navigation is particularly useful if a 3D model of a patient exists and the position and orientation of a tracked instrument (i.e. an instrument with an EM sensor) can be shown in the 3D model during treatment. As known in the art, a 3D model of a patient can be obtained from pre-operative images for instance by means of tomography, for instance using magnetic resonance imaging (MRI). To allow proper alignment of the coordinate system of a 3D model and a real object, for instance a living patient, registration is needed. The EM navigation (i.e. the EM tracking system) as capable to determine the position and orientation of localizers in a coordinate system of the EM navigation system. Registration allows the show the position and orientation of the localizers in real-time images of for instance a patient. Real time images may be obtained with different modalities, for instance a computertomograph or an optical system such as an endoscope that can capture optical images. Likewise, the position and orientation of the localizers can be displayed in 3D images that are reconstructed prom the preoperatively acquired 3D data. In order to track the position and orientation of instruments, localizers are arranged on or in an instrumented to be tracked. Therefore it is possible to display an instrument's position and orientation in real time or reconstructed images. In particular, renderings (i.e. reconstructed 3D images) of an instrument can be super imposed onto reconstructed or real-time images of a patient's anatomy.

One way to achieve registration is photo registration. Photo registration prior to a surgery using electromagnetic navigation refers to the process of incorporating pre-operative images, typically in the form of computer tomography scans or magnetic resonance imaging scans, into a surgical navigation system that utilizes electromagnetic technology, for instance an EM tracking system.

The steps involved in the photo registration process typically are:

### a. Acquisition of Pre-Operative Images

Before the surgery, a patient undergoes tomographic imaging using computer tomography or magnetic resonance imaging, which capture detailed images of the anatomy relevant to the planned surgery.

### 2. Creation of a Digital 3D Model

The acquired images are converted into a digital 3D model using specialized software. This model accurately represents the patient's anatomy and serves as a reference for the surgical navigation system.

### 3. Registration

The next step involves aligning the digital 3D model with the patient's actual anatomy. Electromagnetic sensors placed on the patient's body serve as reference points for the tracking system to precisely detect and track the patient's position and orientation. The electromagnetic sensors placed on the patient's body are typically part of a patient localizer that is attached to the patient.

### 4. EM Tracking System Calibration

The electromagnetic tracking system needs to be calibrated to establish a relationship between the position of the sensors and the corresponding location on the digital 3D model. This calibration allows the system to accurately overlay the digital model onto the patient's anatomy.

### 5. Overlaying the Digital 3D Model

After calibration of the EM tracking system, the digital 3D model is superimposed onto the patient's real-time anatomy. This allows the surgeon to visualize and track instruments for navigating tracked instruments through the patient's body during the surgery using the guidance provided by the registered images.

The step of EM tracking system calibration is a crucial step in the photo registration process that ensures accurate tracking and alignment of the EM tracking system with the patient's anatomy. The calibration step involves:
i. Taking a stereoscopic or 3D image of the patient and the patient localizer with a stereoscopic or a 3D camera. The 3D camera may be a time-of-flight (ToF) camera.
ii. The image taken by the camera is processed to generate a point cloud. The point cloud is a dense collection of 3D data points that represent the patient's outer appearance in a 3D image together with the patient localizer.
iii. The point cloud generated from the 3D images taken by the camera is than aligned with the patient's anatomy as represented in the pre-operative, digital 3D model.
iv. Once the point cloud generated from the 3D images taken by the camera and the patient's anatomy as represented in the pre-operative 3D model are aligned, the relationship between the positions and orientations of the anatomic structures as represented in the 3D model and the EM tracking system's coordinate system can be established.
v. The calibration process may involve additional steps to optimize the accuracy of the alignment. This may include fine-tuning the registration based on known anatomical landmarks or performing iterative adjustments to improve the system's accuracy. Once the calibration is completed, the accuracy and reliability of the EM tracking system are verified and validated. This may involve conducting calibration tests and quality control checks to ensure the system's performance meets the intended accuracy requirements.

Methods and systems for an image guided instrument navigation are inter alia known from U.S. Pat. No. 10,568,713 B2, US 2010/0249506 A1, WO 2021/069745 and U.S. Pat. No. 9,641,808 B2.

During an operation-for instance a surgery-, a patient may move and thus proper registration may be affected. In particular the vertebra of a spine are moving with respect to one another is the patient changes its posture. Therefore, it is not sufficient to track a patient's position by means of a patient localizer for maintaining proper registration of the individual vertebra.

So far, navigation accuracy is usually limited to body regions adjacent to a vertebra body that holds a patient localizer. Especially, when preoperative image data is used which is usually taken in supine position, while intraoperative patient positioning is usually in prone position. Even with intraoperative image data movement of the vertebra bodies relative to each other decreases the accuracy, since only one patient localizer is fixed to one vertebra body.

The invention includes the recognition that the use of preoperatively obtained image data and a 3D model derived therefrom as a basis for navigation does not necessarily reflect an actual 3D configuration of e.g. a person. This image data may differ from the actual situation in the operation room, due to patient positioning.

To mitigate this problem, a patient registration may by carried out intraoperatively, either with intraoperative 3D imaging or with Fluoroscopy imaging representing the correct alignment.

However, even if Intraoperative image data is used as a basis for navigation, this image data gets invalidated with intraoperative movement of the vertebra bodies, for instance after cage placement, spinal realignment) or the like

To mitigate this problem, it is proposed to track the movement of single vertebra bodies intraoperatively and manipulate/augment image data to keep its validity throughout the surgical process.

In particular, it is proposed to
i. equip multiple vertebra bodies with patient localizers,
ii. segment the image data into vertebra body areas wherein segmentation may be performed manually or automatically, for instance by means of a segmenting neural network, and
iii. instead of or in addition to generating and storing a single, global coordinate system transformation during the patient registration process, generate and store an individual coordinate system transformation for each localizer or vertebra body, respectively.

This allows to reflect intraoperative movement of the predefined areas of the image data e.g., after cage placement by applying the individual coordinate system transformations of the individual body members, for instance the individual vertebra bodies.

In particular, a method is proposed that comprises the steps of:
i. generating a 3D model of a mammal form preoperatively obtained tomographic image data,
ii. segmenting the 3D model for obtaining multiple 3D model segments where-in at least some 3D model segments represent rigid body parts, in particular integral bone parts such as individual vertebra bodies or bone fragments, that may move with respect to each other,
iii. generating individual coordinate system transformations using a navigation system for the image guided navigation by way of registration for at least some of the 3D model segments
iv. intraoperatively tracking the position and orientation of a plurality of individual body parts of the mammal by means of the navigation system,
v. adapting a visualization of the 3D model in accordance with the respectively tracked positions and orientations of the individual body parts so as to visualize the individual body parts with their actual position and orientation relative to each other in the visualization of the 3D model,
vi. wherein the adaptation of the visualization of the 3D model comprises applying the individual coordinate system transformations to the respective tracked positions and orientations of the individual body parts, and
vii. displaying the adapted visualization of the 3D model.

An adapted visualization of the 3D model may be generated using a generative neural network that is trained to generate 3D models.

In some embodiments, the navigation system is an electromagnetic navigation system.

According to one non-limiting embodiment, the generation of individual coordinate system transformations using a navigation system for the image guided navigation by way of registration comprises intraoperative recording of image data by way of intraoperative 3D imaging or fluoroscopy imaging.

Instead of or in addition to using a navigation system for the image guided navigation, the generation of individual coordinate system transformations may comprise
i. a segmentation of the intraoperatively recorded images as represented by the intraoperatively recorded image data and
ii. performing registration between 3D model segments of the 3D model and corresponding segments of the intraoperatively recorded images based on the image data.

The method, in some embodiments, further comprises attaching patient localizers to a plurality of rigid body parts, in particular to a plurality of vertebra bodies. The patient localizers are, for example, Kirschner-wires or Jamshidi-needles that each comprise a localizer coil.

For automatically segmenting the 3D model or the intraoperatively obtained images, a segmenting neural network may be used that is trained wit image data representing individual rigid body parts, for instance vertebra bodies.

Further aspects of the invention will become apparent from the following description of some of the embodiments of the invention referring to the figures.

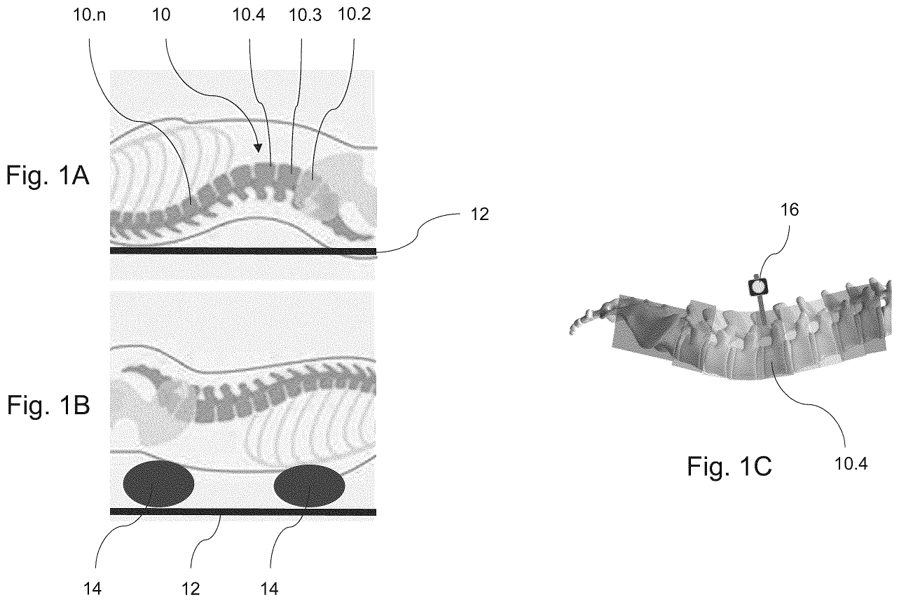

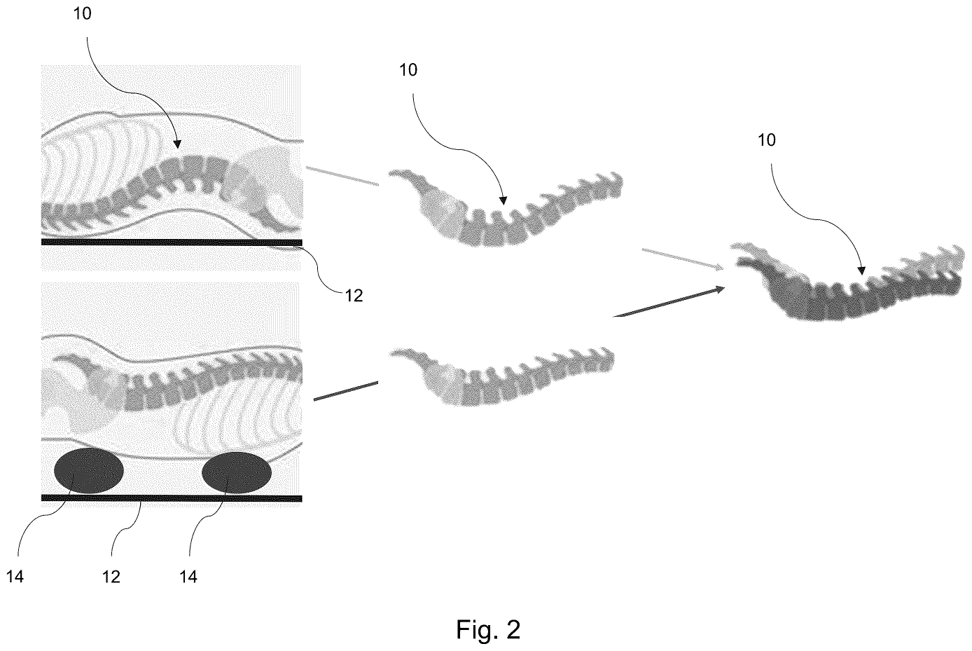

FIG. 1A illustrates the shape of a spine 10 when a patient is place in a supine posture on a flat table 12. This posture may be used for obtaining preoperative image data used for generating a 3D Model.

FIG. 1B illustrates the shape of the spine 10 when the patient is placed on cushions 14 and thus has a prone posture that is typical for a posture during surgery, i. e. an intraoperative posture.

When comparing FIG. 1A and FIG. 1B it is apparent that the shape of the spine 10 depends to a large degree on the posture of the patient and may vary between a preoperative state used for obtaining preoperative image data and an intraoperative state.

FIG. 1C illustrates a spine 10 with individual vertebra bodies 10.1., 10.2 ... 10.n. One vertebra body 10.4 is equipped with a patient localizer 16. A correct registration between the actual position of the vertebra body 10.4 and a 3D model derived from the preoperatively obtained image data is only given for the particular vertebra body 10.4. FIG. 1C further illustrates how the accuracy of the registration decreases for vertebra bodies 10.n depending on their distance from the vertebra body 10.4 that is equipped with the patient localizer 16.

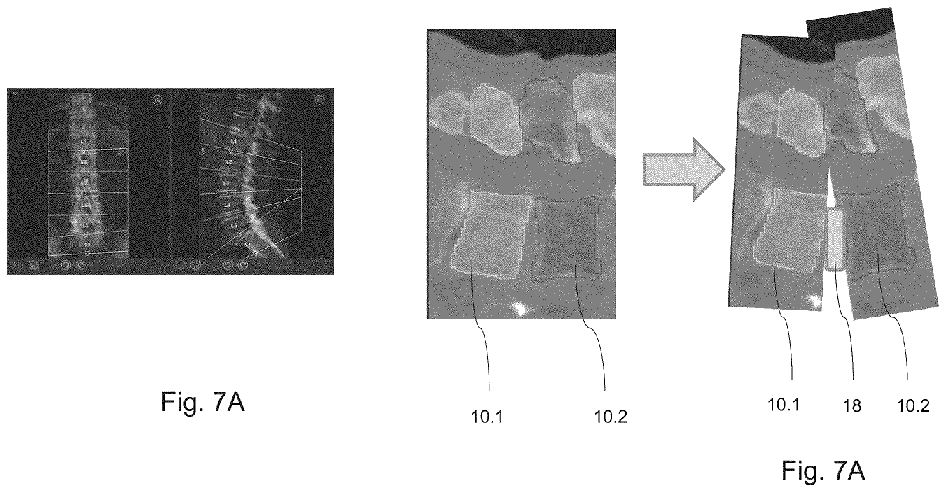

FIG. 2 similarly illustrates the mismatch due to different shapes of the spine 10 before and during an operation. It is noted, that during an operation, the relative position and orientation of vertebra bodies may be changed by surgery, for instance if a cage 18 is placed between two vertebra bodies as illustrated in FIG. 7B.

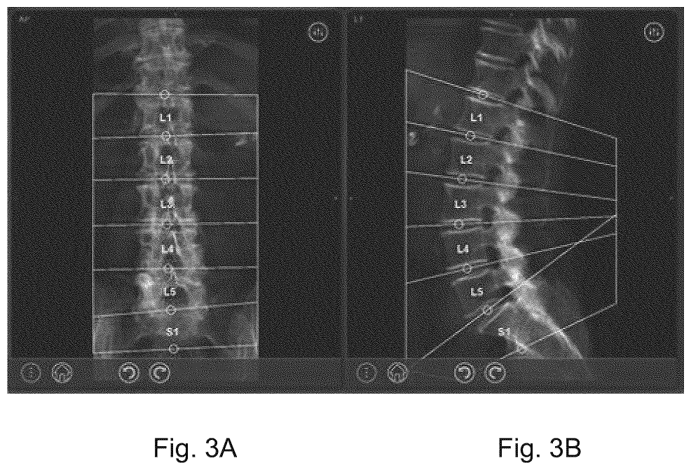

FIG. 3A and B are showing two different fluoroscopic images that are intraoperatively obtained wherein the two images are segmented such that each segment of the fluoroscopic image represents a single, individual vertebra body. Segmentation of intraoperatively obtained images may be performed manually. Alternatively, a segmenting neural network may be used for automatic segmentation of the images. The segmenting neural network may be trained with image data representing fluoroscopic images of individual vertebra bodies.

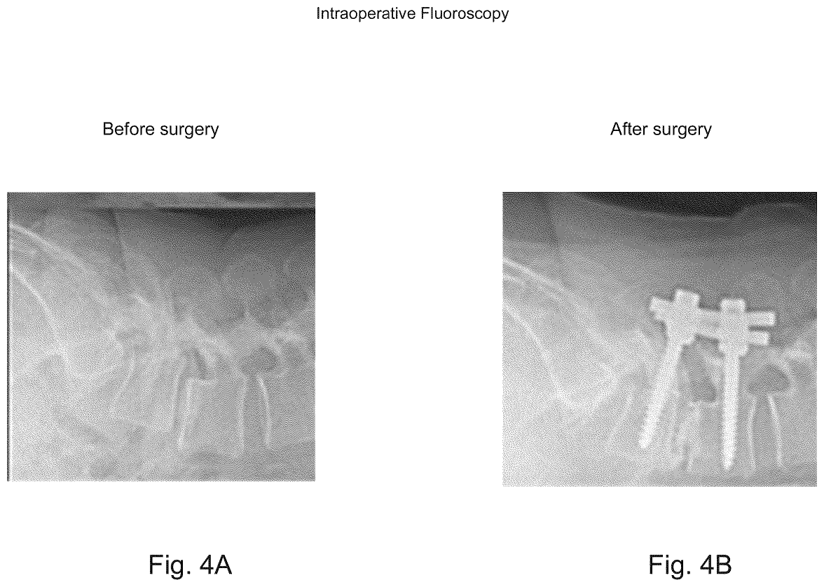

FIG. 4A and B show intraoperatively obtained fluoroscopic images of vertebra bodies. FIG. 4A is a fluoroscopic image that is obtained prior to surgery while FIG. 4B is fluoroscopic image obtained after surgery. It is apparent, that vertebra bodies 10.1 and 10.2 have changed their relative position.

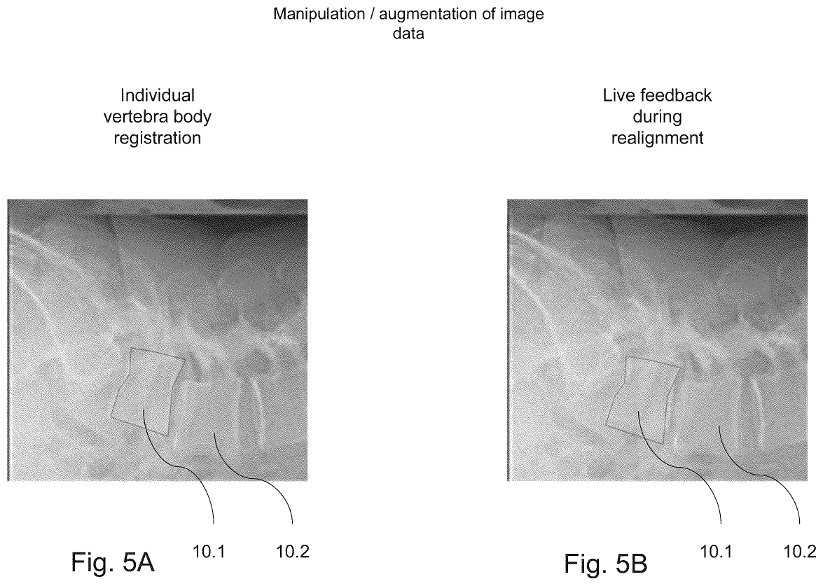

FIG. 5A and B illustrate the effect of individual vertebra body registration due to image segmentation, vertebra bodies 10.1 and 10.2 are individually recognized and thus can be tracked intraoperatively. This can be used to generate augmented images as shown in FIG. 5A and B that reflect the actual position of individual vertebra bodies 10.1 and 10.2 before and after relative movement of the vertebra bodies 10.1 and 10.2.

In order to allow a live tracking of the individual vertebra bodies, each vertebra body may be equipped with an individual patient localizer 16; see FIG. 6. According to one non-limiting embodiment, the patient localizers are navigated Kirschner-wires that each comprise at least one localizer coil.

The individual registration of rigid body parts, in particular the individual registration of individual vertebra bodies allows to reflect intraoperative movement of predefined areas of the image data during surgery. This is illustrated in FIG. 7B. FIG. 7B further illustrates the image segmentation that results in individually recognized vertebra bodies 10.3 and 10.4. FIG. 7A corresponds to FIG. 3 and illustrates how image segmentation may be performed on intraoperatively obtained fluoroscopic images.

### REFERENCE NUMERALS

10 spine
10.1, 10.2 ... 10.n vertebra body
12 table
14 cushion
16 patient localizer
18 cage

The embodiments of the invention described above are intended to be merely exemplary; numerous variations and modifications will be apparent to those skilled in the art. All such variations and modifications are intended to be within the scope of the present invention as defined in any appended claims.

What is claimed is:

1. A method of image guided navigation, comprising the steps of
(a) generating a 3D model of a mammal form preoperatively obtained tomographic image data,
(b) segmenting the 3D model for obtaining multiple 3D model segments wherein at least some 3D model segments represent rigid body parts, in particular integral bone parts such as individual vertebra bodies or bone fragments, that may move with respect to each other,
(c) generating individual coordinate system transformations using a navigation system for the image guided navigation by way of registration for at least some of the 3D model segments
(d) intraoperatively tracking the position and orientation of a plurality of individual body parts of the mammal by means of the navigation system,
(e) adapting a visualization of the 3D model in accordance with the respectively tracked positions and orientations of the individual body parts so as to visualize the individual body parts with their actual position and orientation relative to each other in the visualization of the 3D model, wherein the adaptation of the visualization of the 3D model comprises applying the individual coordinate system transformations to the respective tracked positions and orientations of the individual body parts, and
(f) displaying the adapted visualization of the 3D model.

2. The method of claim 1, wherein the navigation system is an electromagnetic navigation system.

3. The method of claim 1, wherein the generation of individual coordinate system transformations using a navigation system for the image guided navigation by way of registration comprises intraoperative recording of image data by way of intraoperative 3D imaging or fluoroscopy imaging.

4. The method of claim 2, wherein the generation of individual coordinate system transformations using a navigation system for the image guided navigation by way of registration comprises intraoperative recording of image data by way of intraoperative 3D imaging or fluoroscopy imaging.

5. The method of claim 3, comprising segmentation of the intraoperatively recorded images as represented by the intraoperatively recorded image data and performing registration between 3D model segments of the 3D model and corresponding segments of the intraoperatively recorded images.

6. The method of claim 4, comprising segmentation of the intraoperatively recorded images as represented by the intraoperatively recorded image data and performing registration between 3D model segments of the 3D model and corresponding segments of the intraoperatively recorded images.

7. The method of claim 1, further comprising attaching patient localizers to a plurality of rigid body parts, in particular to a plurality of vertebra bodies.

8. The method of claim 2, further comprising attaching patient localizers to a plurality of rigid body parts, in particular to a plurality of vertebra bodies.

9. The method of claim 3, further comprising attaching patient localizers to a plurality of rigid body parts, in particular to a plurality of vertebra bodies.

10. The method of claim 4, further comprising attaching patient localizers to a plurality of rigid body parts, in particular to a plurality of vertebra bodies.

11. The method of claim 5, further comprising attaching patient localizers to a plurality of rigid body parts, in particular to a plurality of vertebra bodies.

12. The method of claim 6, further comprising attaching patient localizers to a plurality of rigid body parts, in particular to a plurality of vertebra bodies.

13. The method of claim 7, wherein the patient localizers are Kirschner-wires or Jamshidi-needles that each comprise a localizer coil.

14. The method of claim 8, wherein the patient localizers are Kirschner-wires or Jamshidi-needles that each comprise a localizer coil.

15. The method of claim 9, wherein the patient localizers are Kirschner-wires or Jamshidi-needles that each comprise a localizer coil.

16. The method of claim 10, wherein the patient localizers are Kirschner-wires or Jamshidi-needles that each comprise a localizer coil.

17. The method of claim 11, wherein the patient localizers are Kirschner-wires or Jamshidi-needles that each comprise a localizer coil.

18. The method of claim 12, wherein the patient localizers are Kirschner-wires or Jamshidi-needles that each comprise a localizer coil.
